(19) United States
(12) Patent Application Publication
(10) Pub. No.: US 2008/0226753 A1
(43) Pub. Date: Sep. 18, 2008

(54) Method of Reducing Insect Resistant Pests in Transgenic Crops

(75) Inventor: Daniel J. Cosgrove, Johnston, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., Johnston, IA (US)

(21) Appl. No.: 10/599,307

(22) PCT Filed: Mar. 29, 2005
(86) PCT No.: PCT/US05/10523

§ 371 Date: Sep. 25, 2006

Related U.S. Application Data
(60) Provisional application No. 60/557,815, filed on Mar. 29, 2004.

(51) Int. Cl. A01N 65/00; A01N 37/08; A01N 43/40; A01N 33/26; A01N 43/647; A01N 57/12; A01N 43/36; A01N 43/56; A01N 37/44; A61K 38/16; A01N 43/04; A01P 7/00

(52) U.S. Cl. 424/725; 514/530; 514/277; 514/611; 514/359; 514/143; 514/408; 514/406; 514/664; 514/561; 514/12; 514/44

(57) ABSTRACT

The present invention discloses Resistance Management (RM) practices that are critical to safeguarding Bacillus thuringiensis as a natural resource and to sustaining genetically modified crops that express Bt toxins for managing ECB and WCRW. The methods involve blending seed transformed with a nucleic acid encoding a different pesticidal protein, where both proteins target the same pest, but use different modes of pesticidal action. The seed can be also treated with pesticidal agents.

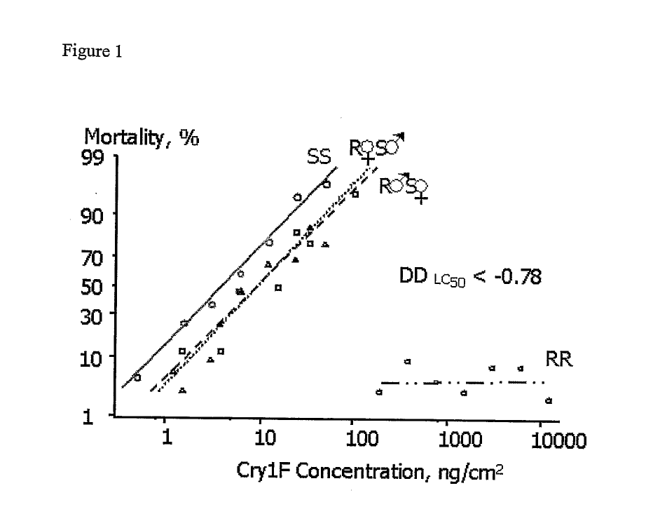

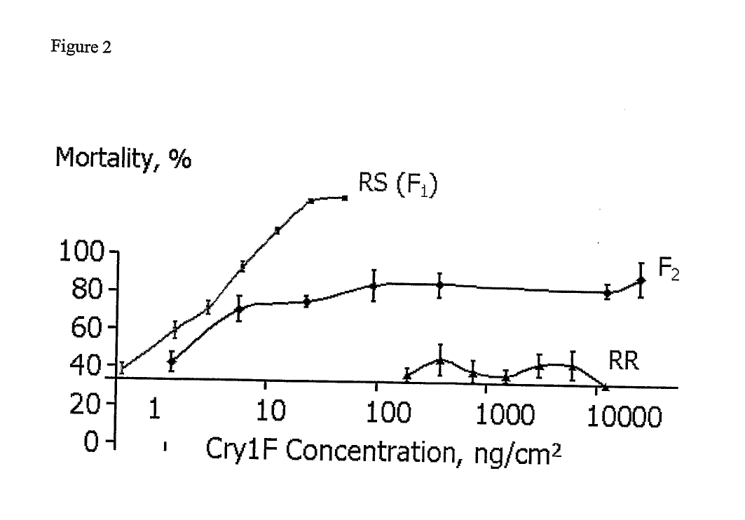

### FIELD OF THE INVENTION

The present invention relates generally to the control of pests that cause damage to crop plants, and in particular to corn plants, by their feeding activities directed to root damage, and more particularly to the control of such plant pests by combining a crop plant seed comprising a first one or more transgenes which express one or more proteins toxic to said plant pests in a mixture of seeds with a second one or more transgenes which express one or more proteins toxic to said plant pests. The first one or more transgenes and the second one or more transgenes are each, respectively, insecticidal to the same target insect and bind without competition to different binding sites in the gut membranes of the target insect. In addition, the treatment of such seed with a chemical or peptide associated pesticide prior to planting the seed is also disclosed.

### BACKGROUND OF THE INVENTION

Insects, nematodes, and related arthropods annually destroy an estimated 15% of agricultural crops in the United States and even more than that in developing countries. In addition, competition with weeds and parasitic and saprophytic plants account for even more potential yield losses.

Some of this damage occurs in the soil when plant pathogens, insects and other such soil borne pests attack the seed after planting. In the production of corn, for example, much of the rest of the damage is caused by rootworms—insect pests that feed upon or otherwise damage the plant roots; and by cutworms, European corn borers, and other pests that feed upon or damage the above ground parts of the plant. General descriptions of the type and mechanisms of attack of pests on agricultural crops are provided by, for example, Metcalf (1962) in Destructive and Useful Insects: Their Habits and Control, Fourth Edition. (Earlier editions by C. L. Metcalf and W. P. Flint) McGraw-Hill Book Company; New York, San Francisco, Toronto, London.; and Agrios, (1988) in Plant Pathology, 3^{rd} Ed., Academic Press.

Lepidopteran insects cause considerable damage to maize crops throughout North America and the world. One of the leading pests is Ostrinia nubilalis, commonly called the European Corn Borer (ECB). Genes encoding the crystal proteins Cry1A(b) and Cry1A(c) from Bt have been introduced into maize as a means of ECB control. The Cry1 group includes, but is not limited to, Cry1A(a), Cry1A(b) and Cry1A(c). See Hofte et al (1989) Microbiol Rev 53: 242-255. These transgenic maize hybrids have been effective in control of ECB (U.S. Pat. Nos. 6,180,744, 5,689,052 and U.S. publication 2002/013227). Recently, Cry1F expressing maize hybrids have also been developed for control of ECB (Chambers, et al. (1991). J. Bact. 173:3966-3976 and Herman, et al. (2002). J. Agric. Food Chem. 50:7076-7078, U.S. Pat. Nos. 5,691,308, 5,188,960 and WO 99/24581). However, developed resistance to Bt toxins presents a challenge in pest control. See McGaughey et al. (1998) Nature Biotechnology 16: 144-146; Estruch et al. (1997) Nature Biotechnology 15:137-141; Roush et al. (1997) Nature Biotechnology 15 816-817; and Hofte et al (1989) supra.

The primary site of action of Cry1 toxins is in the brush border membranes of the midgut epithelia of susceptible insect larvae such as Lepidopteran insects. Cry1A toxin binding polypeptides have been characterized from a variety of Lepidopteran species. A Cry1A(c) binding polypeptide with homology to an aminopeptidase N has been reported from Manduca sexta, Lymantria dispar, Helicoverpa zea and Heliothis virescens. See Knight et al. (1994) Mol Micro 11: 429-436; Lee et al. (1996) Appl Environ Micro 63: 2845-2849; Gill et al. (1995) J Biol. Chem 270: 27277-27282; and Garczynski et al. (1991) Appl Environ Microbiol 10: 2816-2820.

Another Bt toxin binding polypeptide (BTR1) cloned from M. sexta has homology to the cadherin polypeptide superfamily and binds Cry1A(a), Cry1A(b) and Cry1A(c). See Vadlamudi et al. (1995) J Biol Chem 270(10):5490-4, Keeton et al. (1998) Appl Environ Microbiol 64(6):2158-2165; Keeton et al. (1997) Appl Environ Microbiol 63(9):3419-3425 and U.S. Pat. No. 5,693,491.

A subsequently cloned homologue to BTR1 demonstrated binding to Cry1A(a) from Bombyx mori as described in Thara et al. (1998) Comparative Biochemistry and Physiology, Part B 120:197-204 and Nagamatsu et al. (1998) Biosci. Biotechnol. Biochem. 62(4):727-734.

Other serious insect pests of corn in the midwestern United States are the larval forms of three species of Diabrotica beetles. These include the Western corn rootworm, Diabrotica virgifera virgifera LeConte, the Northern corn rootworm, Diabrotica barberi Smith and Diabrotica barberi Lawrence, and the Southern corn rootworm, Diabrotica undecimpunctata howardi Barber.

Corn rootworms (CRW) overwinter in the egg state in fields where corn was grown the previous season. The eggs hatch from late May through June. If a corn crop is not followed by another corn crop in the subsequent year, the larvae will die. Accordingly, the impact of corn rootworm is felt most directly in areas where corn is systematically followed by corn, as is typical in many areas of the midwestern United States.

There is evidence of the emergence of a new race of corn rootworm which ovipositions its eggs for overwintering onto adjacent soybean plants. The most common practice in the mid-western United States has been for fields to be rotated annually with corn, followed the next year with soybeans, in order to manage the development of an epidemic of corn rootworm pressure on fields of corn. While this strategy overall has been successful in reducing the corn rootworm feeding pressure on corn in many areas, the evolutionary emergence of this new race of corn rootworm creates a problem which was not anticipated and which could not have been easily foreseen. This new race, which preferentially deposits its eggs onto soybean fields, provides an unintended feeding pressure on the next year's intended corn crop in the field in which soybeans were grown the previous year, and the subsequent requirement for insecticidal control measures which adds unintended cost to the farmer in the form of additional labor for spraying and additional costs of goods, further reducing the return to the farmer on his/her investment in the crop and harvest.

The western corn rootworm (WCRW), D. virgifera virgifera, is a widely distributed pest of corn in North America, and in many instances, chemical insecticides are indiscriminately used to keep the numbers of rootworms below economically damaging levels. In order to assist in the reduction of chemical insecticides used in treatments to control the rootworm population in crop fields, transgenic lines of corn have been developed which produce one of a number of amino acid sequence variants of an insecticidal protein produced naturally in the bacterium Bacillus thuringiensis. One such protein, generally referred to as Cry3Bb, has recently been modified by English et al., in U.S. Pat. No. 6,023,013 and related patents and applications, to contain one or more amino acid sequence variations which, when tested in insect bioassay against the corn rootworm, demonstrates from about seven (7) to about ten (10) fold increase in insecticidal activity when compared to the wild type amino acid sequence. Another Bt toxin that has been found to be effective in transgenic plants for the control of WCRW is Cry34/35 (U.S. Pat. Nos. 6,548,291, 6,083,499, 6,128,180, 6,624,145 and 6,677,148).

As indicated above, one concern is that resistant ECB and WCRW will emerge. One strategy for combating the development of resistance is to select a recombinant corn event which expresses high levels of the insecticidal protein such that one or a few bites of a transgenic corn plant would cause at least total cessation of feeding and subsequent death of the pest.

Another strategy would be to combine a second ECB or WCRW specific insecticidal protein in the form of a recombinant event in the same plant or in an adjacent plant, for example, another Cry protein or alternatively another insecticidal protein such as a recombinant acyl lipid hydrolase or insecticidal variant thereof (WO 01/49834). Preferably the second toxin or toxin complex would have a different mode of action than the first toxin, and preferably, if receptors were involved in the toxicity of the insect to the recombinant protein, the receptors for each of the two or more insecticidal proteins in the same plant or an adjacent plant would be different so that if a change of function of a receptor or a loss of function of a receptor developed as the cause of resistance to the particular insecticidal protein, then it should not and likely would not affect the insecticidal activity of the remaining toxin which would be shown to bind to a receptor different from the receptor causing the loss of function of one of the two insecticidal proteins cloned into a plant. Accordingly, the first one or more transgenes and the second one or more transgenes are each, respectively insecticidal to the same target insect and bind without competition to different binding sites in the gut membranes of the target insect.

Still another strategy would combine a chemical pesticide with a pesticidal protein expressed in a transgenic plant. This could conceivably take the form of a chemical seed treatment of a recombinant seed which would allow for the dispersal into a zone around the root of a pesticidally controlling amount of a chemical pesticide which would protect root tissues from target pest infestation so long as the chemical persisted or the root tissue remained within the zone of pesticide dispersed into the soil.

Because of concern about the impact of chemical pesticides on public health and the health of the environment, significant efforts have been made to find ways to reduce the amount of chemical pesticides that are used. Recently, much of this effort has focused on the development of transgenic crops that are engineered to express insect toxicants derived from microorganisms. For example, U.S. Pat. No. 5,877,012 to Estruch et al. discloses the cloning and expression of proteins from such organisms as Bacillus, Pseudomonas, Clavibacter and Rhizobium into plants to obtain transgenic plants with resistance to such pests as black cutworms, armyworms, several borers and other insect pests. Publication WO/EP97/07089 by Privalle et al. teaches the transformation of monocotyledons, such as corn, with a recombinant DNA sequence encoding peroxidase for the protection of the plant from feeding by corn borers, earworms and cutworms. Jansens et al. (1997) Crop Sci., 37(5): 1616-1624, reported the production of transgenic corn containing a gene encoding a crystalline protein from Bacillus thuringiensis (Bt) that controlled both generations of ECB. U.S. Pat. Nos. 5,625,136 and 5,859,336 to Koziel et al. reported that the transformation of corn with a gene from B. thuringiensis that encoded for delta-endotoxins provided the transgenic corn with improved resistance to ECB. A comprehensive report of field trials of transgenic corn that expresses an insecticidal protein from B. thuringiensis has been provided by Armstrong et al., in Crop Science, 35(2):550-557 (1995).

Another alternative to the conventional forms of pesticide application is the treatment of plant seeds with pesticides. The use of fungicides or nematicides to protect seeds, and young roots and shoots from attack after planting and sprouting, and the use of low levels of insecticides for the protection of, for example, corn seed from wireworm, has been used for some time. Seed treatment with pesticides has the advantage of providing for the protection of the seeds, while minimizing the amount of pesticide required and limiting the amount of contact with the pesticide and the number of different field applications necessary to attain control of the pests in the field.

Other examples of the control of pests by applying insecticides directly to plant seed are provided in, for example, U.S. Pat. No. 5,696,144, which discloses that ECB caused less feeding damage to corn plants grown from seed treated with a 1-arylpyrazole compound at a rate of 500 g per quintal of seed than control plants grown from untreated seed. In addition, U.S. Pat. No. 5,876,739 to Turnblad et al. (and its parent, U.S. Pat. No. 5,849,320) disclose a method for controlling soil-borne insects which involves treating seeds with a coating containing one or more polymeric binders and an insecticide. This reference provides a list of insecticides that it identifies as candidates for use in this coating and also names a number of potential target insects.

Although recent developments in genetic engineering of plants have improved the ability to protect plants from pests without using chemical pesticides, and while such techniques such as the treatment of seeds with pesticides have reduced the harmful effects of pesticides on the environment, numerous problems remain that limit the successful application of these methods under actual field conditions. Accordingly, it would be useful to provide an improved method for the protection of plants, especially corn plants, from feeding damage by pests. It would be particularly useful if such a method would reduce the required application rate of conventional chemical pesticides, and also if it would limit the number of separate field operations that were required for crop planting and cultivation.

In addition, it would be useful to have a method of deploying a transgenic refuge required by the regulatory agencies in a field of transgenic crops instead of peripheral to a field of transgenic crops.

### SUMMARY OF THE INVENTION

The present invention discloses a method of reducing the development of resistant pests in a field of transgenic pest resistant crops comprising the steps of:
a) blending seed of a first transgenic pest resistant crop with seed of a second transgenic pest resistant crop to provide a seed mixture wherein said first pest resistant crop and said second pest resistant crop are pesticidal to the same target pest but through a different mode of pesticidal action; and
b) planting said seed mixture in a field wherein said seed mixture consists of from about 100% to about 50% of said first transgenic pest resistant crop and of from about 100% to about 50% of said second transgenic pest resistant crop.

Target pests of the present invention include ECB and WCRW. The present invention utilizes different modes of pesticidal action which comprises toxin binding without competition to different binding sites in the gut membranes of the same target pest. Seed of the present invention is further treated with a pesticidal agent selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, and carbaamates. Transgenes useful in the present invention include those producing a Cry1F protein, a Cry 1A(b) protein, a Cry 34/35 protein and Cry 3 protein.

In addition, the present invention discloses a method of reducing the development of resistant pests in a field of transgenic pest resistant crops comprising the steps of:
a) blending seed of a first transgenic pest resistant crop which contains one or more transgenes having pesticidal activity against a first target pest wherein said one or more transgenes are pesticidal to said first target pest through different modes of pesticidal action, with seed of a second transgenic pest resistant crop which contains one or more transgenes having pesticidal activity against a second target pest wherein said one or more transgenes are pesticidal to said second target pest through different modes of pesticidal action, to provide a seed mixture wherein said first pest resistant crop and said second pest resistant crop are pesticidal to different target pests; and
b) planting said seed mixture in a field wherein said seed mixture consists of from about 100% to about 50% of said first transgenic pest resistant crop and of from about 100% to about 50% of said second transgenic pest resistant crop.

Particular target pests of interest include ECB and WCRW, wherein said different modes of pesticidal action comprise binding without competition to different binding sites in the gut membranes of the target pests.

The transgenic seed of the present invention may also contain a herbicide resistance gene selected from the group consisting of GAT and EPSPS.

Embodiments of the invention also include a method for deploying a transgenic pest resistant refuge crop into a field of a transgenic pest resistant crop comprising the steps of:
a) blending seed of a transgenic pest resistant refuge crop with seed of a transgenic pest resistant crop to provide a seed mixture wherein said pest resistant refuge crop and said pest resistant crop are pesticidal to the same target pest but through a different mode of pesticidal action; and
b) planting said seed mixture in a field wherein said seed mixture consists of from about 100% to about 50% of said transgenic pest resistant refuge crop and of from about 100% to about 50% of said transgenic pest resistant crop are also disclosed. The method for deploying a transgenic pest resistant refuge crop into a field of a transgenic pest resistant crop contemplates the use of multiple transgenes.

In particular, the embodiments include a method for deploying a transgenic pest resistant refuge crop into a field of a transgenic pest resistant crop comprising the steps of:
a) blending seed of a transgenic pest resistant refuge crop which contains one or more transgenes having pesticidal activity against a first target pest wherein said one or more transgenes are pesticidal to said first target pest through different modes of pesticidal action, with seed of a transgenic pest resistant crop which contains one or more transgenes having pesticidal activity against a second target pest wherein said one or more transgenes are pesticidal to said second target pest through different modes of pesticidal action, to provide a seed mixture wherein said pest resistant refuge crop and said pest resistant crop are pesticidal to different target pests; and
b) planting said seed mixture in a field wherein said seed mixture consists of from about 100% to about 50% of said transgenic pest resistant refuge crop and of from about 100% to about 50% of said transgenic pest resistant crop is disclosed.

### DETAILED DESCRIPTION OF THE INVENTION

As used herein, the term “corn” means Zea mays or maize and includes all plant varieties that can be bred with corn, including wild maize species.

As used herein, the term “comprising” means “including but not limited to”.

As used herein, the terms “pest”, “pesticide”, and “pesticidal” are meant to be interchangeable and inclusive of the following terms: for example, insect, insecticide, and insecticidal when referring to an insect pest; or with the terms, for example, nematode, nematicide, and nematicidal when referring to a nematode pest; or with acaric, acaricide, and acaricidal when referring to an acaric pest; or with fungus or fungal, fungicide, and fungicidal or equivalent terms such as mycotic, and mycocidal when referring to fungal or related pests; or with plant or herb, planticide or herbicide, or planticidal or herbicidal when referring to a plant or a herb pest.

As used herein, the term “transgenic refuge” refers to the requirement of a resistance management plan for reducing or eliminating the likelihood of development of resistance to one or more insecticides that are either present within a recombinant plant or present adjacent to one or more parts or tissues of a plant.

As used herein, the terms “pesticidal effect” and “pesticidal activity”, or “activity” refer to a toxic effect against a pest. The terms “activity against (one or more) pests”, also have the same meaning. When it is said that a seed or plant is “protected against feeding damage by one or more pests”, it is meant that such seed or plant possesses a feature having direct or indirect action on one or more pests that results in reduced feeding damage by such pest or pests on the seeds, roots, shoots and foliage of plants having such feature as compared to the feeding damage caused under the same conditions to plants not having such feature. Such direct or indirect actions include inducing death of the pest, repelling the pest from the plant seeds, roots, shoots and/or foliage, inhibiting feeding of the pest on, or the laying of its eggs on, the plant seeds, roots, shoots and/or foliage, and inhibiting or preventing reproduction of the pest.

The term “insecticidal activity” has the same meaning as pesticidal activity, except it is limited to those instances where the pest is an insect. Except where specifically noted, when the term “pesticide” is used herein, that term refers to a chemical pesticide that is supplied externally to the seed, and it is not meant to include active agents that are produced by the particular seed or the plant that grows from the particular seed. However, the terms “pesticidal activity” and “insecticidal activity” can be used with reference to the activity of either, or both, an externally supplied pesticide and/or an agent that is produced by the seed or the plant.

The European Corn Borer (ECB), Ostrinia nubilalis (Hubner), is one of the most destructive pests of maize in the U.S. Management of ECB using genetically modified (GM) maize varieties has been successfully practiced since 1996. Recently, Cry1F expressing maize hybrids have been developed for control of ECB (Chambers, et al. (1991) supra; Herman, et al. (2002), supra; U.S. Pat. Nos. 5,691,308, 5,188,960 and WO 99/24581). Cry 1A(b) expressing maize have also been developed for the control of ECB (U.S. Pat. Nos. 6,180,774, 5,689,052, and U.S. patent application publication 2002/013227).

Resistance Management (RM) practices are critical to safeguard Bacillus thuringiensis as a natural resource and sustain genetically modified corn expressing Bt toxins as a suitable method for ECB and WCRW management. A useful tool in developing RM strategies is to develop laboratory selected colonies that exhibit high levels of resistance to a particular toxin. The availability of selected strains allows determination of the genetic expression of resistance (i.e., dominant vs. recessive, autosomal vs. sex-linked) and whether or not the resistance mechanism is specific for a given toxin. In addition, the availability of resistant strains will allow estimation of the particular resistance allele frequency in the field, and provides a tool to identify the biochemical and physiological basis of resistance and a means to develop molecular probes to monitor the evolution of resistance in the field.

One feature of the present invention is a seed of a transgenic corn plant. As used herein, the terms “transgenic corn plant” mean a corn plant or progeny thereof derived from a transformed corn plant cell or protoplast, wherein the plant DNA contains an introduced exogenous DNA molecule not originally present in a native, non-transgenic plant of the same strain.

It is preferred that the seed contains an exogenous gene derived from a strain of Bacillus thuringiensis, and in particular, it is preferred that the exogenous gene is one that encodes an insecticidal δ-endotoxin derived from B. thuringiensis. Such δ-endotoxins are described in U.S. Pat. Nos. 5,691,308, 5,188,960, 6,180,774, 5,689,052, U.S. patent application publication 2002/013227, and PCT publications WO 99/24581, and WO 99/31248.

When the term “transgenic event” is used herein, it is meant to refer to the genetically engineered DNA that is described above, but also to include the protein(s) that are encoded by the modified gene. A transgenic event in a corn seed, or corn plant, therefore, includes the ability to express a protein. When it is said that a “transgenic event has activity against a pest”, it is to be understood that it is the protein that is encoded by the gene that actually has such activity when the protein is expressed and brought into contact with the pest.

The term “transgenic event” is also meant herein to include recombinant plants produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the heterologous DNA. Even after repeated back-crossing to a recurrent parent, the inserted DNA and flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

It is also to be understood that two different transgenic plants can also be mated to produce offspring that contain two independently segregating added, exogenous genes. Selfing of appropriate progeny can produce plants that are homozygous for both added, exogenous genes. Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcox J. ed., American Society of Agronomy, Madison, Wis. (1987).

Transgenic plants known as transgenic events herein derived from the insertion of a DNA sequence designed to express the Cry1A(b) variant protein are designated as transgenic event No. MON810. Transgenic plants known as transgenic events herein derived from the insertion of a DNA sequence designed to express the Cry1F variant protein are designated as transgenic event No. TC1507. Transgenic plants known as transgenic events herein derived from the insertion of a DNA sequence designed to express the Cry3Bb variant protein 11231 are designated as transgenic event No. MON853. Transgenic plants known as transgenic events herein derived from the insertion of a DNA sequence designed to express the Cry3Bb variant protein 11098 are designated as transgenic event No. MON863. Transgenic plants known as transgenic events herein derived from the insertion of a DNA sequence designed to express the Cry34/35 variant protein are designated as transgenic event No. E4497.59.1.22.

It has also been found that a preferred use of the present invention is for reducing pest feeding damage when used in combination with seeds having transgenic events that have certain levels of effectiveness against such pest. To illustrate which levels of effectiveness are preferred, the following example will use the Iowa Root Rating Method (Hills and Peters (1971) J. Econ. Entomol., 64:764-765), which measures corn rootworm feeding damage to corn roots on a 1-6 scale. In the rating, 1=no damage or only a few minor feeding scars; 2=feeding scars evident but no roots eaten off to within 1½ inch of the plant; 3=several roots eaten off to within 1½ inch of the plant, but never the equivalent of an entire node of roots is destroyed; 4=one root node completely destroyed; 5=two root nodes completely destroyed; and 6=three or more root nodes destroyed. A destroyed root is defined as a root that has been pruned to within 1½ inch of the base. Pruned roots do not have to originate from a single node, but all pruned roots must equal the equivalent of a full node to count as a destroyed node.

As used herein, a transgenic event is within the preferred range of effectiveness level against a target pest if that event reduces feeding damage by that pest by a certain amount as compared with the same crop without the transgenic event, but does not prevent substantially all damage by the target pest. For example, if 10% of transgenic corn suffered corn rootworm damage of 4 or higher on the Iowa 1-6 Scale, while 80% of non-transgenic corn suffered damage of 4 or higher, then it could be said that the damage to the transgenic corn was (10/80)×100=12.5% of that of the non-transgenic corn. For the purposes of the present invention, it will be understood that a transgenic event in corn is within the preferred range of effectiveness level if corn having such event suffers from about 5% to about 50% of the damage suffered by non-transgenic corn due to the same pest under the same conditions. It is more preferred that corn having such transgenic event suffers from about 10% to about 40% of the damage suffered by non-transgenic corn by the same pest under the same conditions, even more preferred is damage of from about 15% to about 30%, and yet more preferred is damage of from about 20% to about 30% of the damage suffered by non-transgenic corn by the same pest under the same conditions. As used herein, when the term “about” is used to describe the degree of damage to corn, it is to be understood that the degree of damage can be above or below the limits described by as much as 1% or 2% and still be considered to be within the ranges described. By way of example, a level of 4.5% damage would be regarded as being “about 5%”.

The present invention also includes seeds and plants having more than one transgenic event. Such combinations are referred to as “stacked” transgenic events. These stacked transgenic events can be events that are directed at the same target pest, or they can be directed at different target pests. For example, a seed having the ability to express a Cry 1F protein also has the ability to express at least one other insecticidal protein that is different from a Cry 1F protein such as for example a Cry1A(b) protein. Similarly, a seed having the ability to express a Cry 34/35 protein also has the ability to express at least one other insecticidal protein that is different from a Cry 34/35 protein such as a Cry3 protein.

The seed having the ability to express an insecticidal protein also has a transgenic event that provides herbicide tolerance. Preferably, the transgenic event that provides herbicide tolerance is an event that provides resistance to glyphosate-N-(phosphonomethyl) glycine, including the isopropylamine salt form of such herbicide (WO 02/36782, WO 03/09236, U.S. patent application publications 2003/192072 and 2003/188346.

In the present method, a corn seed having a transgenic event is optionally treated with a pesticide. Pesticides suitable for use in the invention include pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates. Known pesticides within these categories are listed in The Pesticide Manual, 11th Ed., C. D. S. Tomlin, Ed., British Crop Protection Council, Farnham, Surry, UK (1997).

Insecticides that are oxadiazine derivatives are useful in the subject method. The oxadizine derivatives that are preferred for use in the present invention are those that are identified in U.S. Pat. No. 5,852,012. Chloronicotinyl insecticides are also useful in the subject method. Chloronicotinyls that are preferred for use in the subject composition are described in U.S. Pat. No. 5,952,358. Nitroguanidine insecticides are useful in the present method. Such nitroguanidines can include those described in U.S. Pat. Nos. 5,633,375, 5,034,404 and 5,245,040. Pyrrols, pyrazoles and phenyl pyrazoles that are useful in the present method include those that are described in U.S. Pat. No. 5,952,358.

When an insecticide is described herein, it is to be understood that the description is intended to include salt forms of the insecticide as well as any isomeric and/or tautomeric form of the insecticide that exhibits the same insecticidal activity as the form of the insecticide that is described. The insecticides that are useful in the present method can be of any grade or purity that pass in the trade as such insecticide. Other materials that accompany the insecticides in commercial preparations as impurities can be tolerated in the subject methods and compositions, as long as such other materials do not destabilize the composition or significantly reduce or destroy the activity of any of the insecticide components or the transgenic event(s) against the target pest(s). One of ordinary skill in the art of the production of insecticides can readily identify those impurities that can be tolerated and those that cannot.

It has been found that the present method is useful to protect seeds and plants against a wide array of agricultural pests, including insects, mites, fungi, yeasts, molds, bacteria, nematodes, weeds, and parasitic and saprophytic plants.

When an insect is the target pest for the present invention, such pests include but are not limited to: insects of the order Lepidoptera, e.g. Achroia grisella, Acleris gloverana, Acleris variana, Adoxophyes orana, Agrotis ipsilon, Alabama argillacea, Alsophila pometaria, Amyelois transitella, Anagasta kuehniella, Anarsia lineatella, Anisota senatoria, Antheraea pernyi, Anticarsia geminatalis, Archips sp., Argyrotaenia sp., Athetis mindara, Bombyx mori, Bucculatrix thurberiella, Cadra cautella, Chroristoneura sp., cochylis hospes, Colias eurytheme, Corcyra cephalonica, cydia latiferreana, Cydia pomonella, datana integerrima, Dendrolimus superans sibiricus, Desmia funeralis, Diaphania hyalinata, Diaphania nitidalis, Diatraea grandiosells, Diatraea saccharalis, Ennomos subsignaria, Eoruma loftini, Ephestia elutella, Erannis tilaria, Estigmene acrea, Bonogota salubricola, Eupoecilia ambiguella, Euproctis chrysorrhoea, Euxoa messoria, Galleria mellonella, Grapholita molesta, Harristina americana, Heliothis subflexa, Helicoverpa zea, Heliothis virescens, Hemileuca oliviae, Homoeosoma electellum, Hyphantria cunea, Keiferia lycopersicella, Lambina fiscellaria fiscellaria, Lambdina fiscellaria lugubrosa, Leucoma salicis, Lobesia botrana, Loxostege sticticalis, Lymantria dispar, Macalla thyrisalis, Malacosoma sp., Mamestra brassicae, Mamestra configurata, Manduca quinquemaculata, Manduca sexta, Maruca testulalis, Melanchra picta, Operophtera burmata, Orgyia sp., Ostrinia nubilalis, Paleacrita vernata, Papilio cresphontes, Pectinophora gossypiella, Phryganidia californica, Phyllonorcycter blancardella, Pieris napi, Pieris rapae, Plathypena scabra, Platynota flavedana, Platynota stultana, Platyptilia carduidactyla, Plodia interpunctella, Plutella xylostella, Pontia protidice, Psedaletia unipuncata, Pseudoplusia includens, Sabulodes aegrotata, Schizura concinna, Sitotroga cerealella, Spilonta ocellana, Spodoptera sp., Thaumetopoea pityocampa, Tineola bisselliella, Trichoplusia ni, Udea rubigalis, Xylomyges curialis, and Yponomeuta padella.

Also, the embodiments of the present invention may be effective against insect pests including insects selected from the orders Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera, especially Diabrotica virgifera and Lepidoptera. Insect pests of the invention for the major crops include, but are not limited to: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; western corn rootworm, e.g., Diabrotica virgifera virgifera; northern corn rootworm, e.g., Diabrotica longicornis barberi; southern corn rootworm, e.g., Diabrotica undecimpunctata howardi; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; spenophorus maidis, maize billbug; Rhopalosiphum maidis, corn leaf aphid; Aphis maidiradicis, corn root aphid; Blissus leucopterus leucopteris, chinch bug; Melanoplus femurrubrum, redlegged grasshopper; Melanoplus sanguinipes, migratory grasshopper; Delia platura, seedcorn maggot; Agromyza parvicornis, corn blotch leafminer; Anaphothrips obscurus, grass thrips; Solenopsis molesta, thief ant; Tetranychus urticae, twospotted spider mite; Sorghum: Chilo partellus, sorghum stem borer; Spodoptera frugiperda, fall armyworm; Helicoverpa zea, corn earworm; Elasmopalpus lignosellus, leser cornstalk borer; Agrotis subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcan aphid; chinch bug, e.g., Blissus leucopterus leucopterus; Contarinia sorghicola, sorghum midge; Tetranychus cinnabarinus, carmine spider mite; Tetranychus urticae, twospotted spider mite; Wheat: Pseudaletia unipunctata, army work; Spodoptera frugiperda, fall armyworm; Elasmopalpus lignosellus, lesser cornstalk borer; Agrotis orthogonia, pale western cutworm; Oulema melanopus, cereal leaf beetle; Hypera punctata, clover leaf weevil; sourthern corn rootworm, e.g., Diabrotica undecimpunctata howardi; russian wheat aphid; Schizaphis graminum, greenbug; Sitobium avenae, English grain aphid; Melanoplus femurrubrum, redlegged grassphooper; Melanoplus differentialia, differential grasshopper; Melanoplus sanguinipes, migratory grasshopper; Mayetiola destructor, Hessian fly; Sitodiplosis mosellana, wheat midge; Meromyza americans, wheat stem maggot; Delia coarctata, wheat bulb fly; Frankliniella fusca, tobacco thrips; Cephus cinctus, wheat stem sawfly; Aceria tosichella, wheat curl mite; Sunflower: Cylindrocopturus adspersus, sunflower stem weevil; Smicronyx fulus, red sunflower seed weevil; Smicronyx sordidus, gray sunflower seed weevil; Suleima helianthana, sunflower bud moth; Homoeosoma electellum, sunflower moth; Zygogramma exclamationis, sunflower seed midge; Cotton: Heliothis virescens, tobacco budworm; Helicoverpa zea, cotton bollworm; spodoptera exigua, beet armyworm; Pectinophora gossypiella, pink bollworm; boll weevil, e.g., Anthonomus grandis; Aphis gossypii, cotton aphid; Pseudatomoscelis seriatus, cotton fleahopper; Trialeurodes abutiloneus, bandedwinged whitefly; Lygus lineolaris, tarnished plant bug; Melanoplus femurrubrum, redlegged grasshopper; Melanoplus differentialis, differential grasshopper; Thrips tabaci, onion thrips; Frankliniella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; colaspis brunnea, grape colaspis; Lissorhoptrus oryzophilus, rice water weevil; Sitophilus oryzae, rice weevil; Nephotettix nigropictus, rice leafhopper; chinch bug, e.g., Blissus leucopterus leucopterus; Acrosternum hilare, green stink bug; soybean: Pseudoplusia includens, soybean looper; Anticarsia gemmatalis, velvetbean caterpillar; Hypena scabra, green cloverworm; Ostrinia nubilalis, European corn borer; Agrotis ipsilon, black cutworm; Spodoptera exigua, beet armyworm; Heliothis virescens, tobacco budworm; Helicoverpa zea, cotton bollworm; Epilachna varivestis, Mexican been beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Delia platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; chinch bug, e.g., Blissus leucopterus leucopterus; Acrosternum hilare, green stink bug; Euschistuts servus, brown stink bug; Delia platura, seedcorn maggot; Mayetiola destructor, Hessian fly; Petrobia latens, brown wheat mite; Oil Seed Rape: Vrevicoryne brassicae, cabbage aphid; Phyllotreta cruciferae, crucifer flea beetle; Phyllotreta striolata, striped flea beetle; Phyllotreta nemorum, striped turnip flea beetle; Meligethes aeneus, rapeseed beetle; and the pollen beetles Meligethes rufimanus, Meligethes nigrescens, Meligethes canadianus, and Meligethes viridescens; Potato: Leptinotarsa decemlineata, Colorado potato beetle.

Furthermore, embodiments of the present invention may be effective against Hemiptera such as Lygus hesperus, Lygus lineolaris, Lygus pratensis, Lygus rugulipennis Popp, Lygus pabulinus, Calocoris norvegicus, Orthops compestris, Plesiocoris rugicollis, Cyrtopeltis modestus, Cyrtopeltis notatus, Spanagonicus albofasciatus, Diaphnocoris chlorinonis, Labopidicola allii, Pseudatomoscelis seriatus, Adelphocoris rapidus, Poecilocapsus lineatus, Nysius ericae, Nysiusraphanus, Euschistus servus, Nezara viridula, Eurygaster, Coreidae, Pyrrhocoridae, Tinidae, Blostomatidae, Reduviidae, and Cimicidae. Pests of interest also include Araecerus fasciculatus, coffee bean weevil; Acanthoscelides obtectus, bean weevil; Bruchus rufimanus, broadbean weevil; Bruchus pisorum, pea weevil; Zabrotes subfasciatus, Mexican bean weevil; Diabrotica balteata, banded cucumber beetle; Cerotoma trifucata, bean leaf beetle; Diabrotica virgifera, Mexican corn rootworm; Epitrix cucumeris, potato flea beetle; Chaetocnema confinis, sweet potato flea beetle; Hypera postica, alfalfa weevil; Anthonomus quadrigibbus, apple curculio; Sternechus paludatus, bean stalk weevil; Hypera brunnipennis, Egyptian alfalfa weevil; Sitophilus granarius, granary weevil; Craponius inaequalis, grape curculio; Sitophilus zeamais, maize weevil; conotrachelus nenuphar, plum curculiol; Euscepes postfaciatus, West Indian sweet potato weevil; Maladera castanea, Asiatic garden beetle; Rhizotrogus majalis, European chafer; Macrodactylus subspinosus, rose chafer; Tribolium confusum, confused flour beetle; Tenebrio obscurus, dark mealworm; Tribolium castameum, red flour beetle; Tenebrio molitor, yellow mealworn.

Nematodes include plant-parasitic nematodes such as root-knot, cyst, and lesion nematodes, including Heterodera and Globodera spp. such as Globodera rostochiensis and Globodera pailida (potato cyst nematodes); Heterodera glycines (soybean cyst nematode); Heterodera schachtii (beet cyst nematode); and Heterodera avenae (cereal cyst nematode).

The subject pesticides can be applied to a seed as a component of a seed coating. Seed coating methods and compositions that are known in the art are useful when they are modified by the addition of one of the embodiments of the combination of pesticides of the present invention. Such coating methods and apparatus for their application are disclosed in, for example, U.S. Pat. Nos. 5,918,413, 5,891,246, 5,554,445, 5,389,399, 5,107,787, 5,080,925, 4,759,945 and 4,465,017. Seed coating compositions are disclosed, for example, in U.S. Pat. Nos. 5,939,356, 5,882,713, 5,876, 739, 5,849,320, 5,834,447, 5,791,084, 5,661,103, 5,622,003, 5,580,544, 5, 328,942, 5,300,127, 4,735,015, 4,634,587, 4,383,391, 4,372,080, 4,339,456, 4,272,417 and 4,245,432, among others.

The treated seeds of the present invention can be used for the propagation of corn plants in the same manner as conventional treated corn seed. The treated seeds can be stored, handled, sowed and tilled in the same manner as any other pesticide treated seed. Appropriate safety measures should be taken to limit contact of the treated seed with humans, food or feed materials, water and birds and wild or domestic animals.

The words “seed mix refuge strategy” are intended to refer to a means for deploying into a field of crops some percentage of the seeds which sprout and develop into mature refuge plants which contains a different non-competitively binding Bt or other insecticidal protein. (U.S. Pat. Nos. 5,866,784, 5,908,970, and 6,172,281.) At the same time, two modes of action are achieved, assuring the longest possible term for commercial viability and utility of the transgenic crop seeds with a minimal risk to the development of resistant races of insects.

In the regulatory environment that currently exists today, obtaining the approval of an appropriate regulatory agency for commercialization of a recombinant plant generally requires that a percentage of all of the crop that is planted by a particular farmer intending to plant a crop containing a recombinant trait which effects the survival of particular insect pests be planted as a refuge of non-recombinant or non-transgenic crops, or crops which do not contain the ability to inhibit the development and growth of the particular insect pest by the same mode of action.

Advantages of a seed mix deployable refuge strategy over a block refuge strategy includes elimination of the issues around enforcement and compliance, simplicity, and complementarity with block refuge strategies required for other insect resistance traits. The first one or more transgenes and the second one or more transgenes are each, respectively insecticidal to the same target insect and bind without competition to different binding sites in the gut membranes of the target insect. Furthermore by adding a seed treatment to the seed mix deployable refuge strategy, no plants are left unprotected in the field and a third mode of action is unifonnly introduced to function along with the transgenic insect control means.

In a 2004 research poster titled “Characterization of Cry1F resistance in laboratory selected European corn borer, Ostrinia nubilalis (Lepidoptera: Crambidae)”, presented at the 59^{th} ESA-NCB Annual Meeting Kansas City, Mo. March, 2004, Pereira et al. reported that a colony of O. nubilalis was selected in the laboratory for high levels of resistance to Cry1F. This resistant colony was compared with a standard susceptible strain to quantify the resistance level and assess whether there was cross resistance to other relevant Bt toxins. The two colonies were also used to establish reciprocal crosses between resistant (RR) and susceptible (SS) parents, and backcross between RS (F1) progeny and RR parents. The progeny were then bioassayed and compared with both parental populations. The Cry1F resistant strain exhibited more than 3000-fold resistance. No cross resistance to Cry1Ab and Cry9C was observed although a low but significant (6.89 fold) cross resistance to Cry1Ac was detected. Genetic experiments indicate that the resistance is autosomal and almost completely recessive. Backcross of the F1 generation to the resistant parents suggests that a single gene is responsible for the resistance.

The resistant strain used by Pereira et al. (2004), supra, was obtained by selecting a colony originally established from field collections at 10 geographically isolated locations within the central U.S. Corn Belt in 1996, and then in 1998, after the colony had been maintained for 7 generations using standard rearing conditions, selection to Cry1F began. The colony was selected with increasing concentrations of Cry1F incorporated into artificial diet for 30 generations and then maintained at 35 μg/mL Cry1F for approximately 10 generations. In 2001 and 2002 further selection was conducted using truncated Cry1F applied to the surface of artificial diet for 7 days. Maintenance of the Cry1F selected colony was achieved by exposing neonate larvae to 60 ng/cm², which corresponds to the upper limit of the 95% confidence interval of the LC₉₉ for susceptible populations.

Pereira et al. (2004) supra, reported that bioassays on the strains were initially conducted in January of 2003 in the 55th generation. The Cry1F toxin used in the bioassays consisted of a chromatographically purified and proteolytically truncated protein (provided by Dow Agrosciences). ECB were reared using standard techniques (Lewis, et al. (1969) Iowa State J. Sci. 44: 9-14). The susceptibility of neonate ECB to Bt toxins was determined by exposure to varying concentrations of Bt toxin applied on the surface of artificial diet (Marcon, et al. (1999) J Econ. Entomol. 92: 279-285). Dilutions were prepared in 0.1% Triton-X 100; bioassays were conducted in duplicate on 2 dates and included 7 concentrations of purified Bt toxins. Larval mortality was recorded after seven days of exposure. Cross resistance was assessed to Cry1Ab, Cry1Ac, and Cry9C.

To obtain reciprocal crosses between susceptible (SS) and resistant (RR) parents, 300 pupae from each population were sexed and isolated into mating cages so that males from one population (RR) could mate with virgin females from the other (SS), and vice-versa. Cages with males and females of the same population were also arranged so that the resistant (RR), susceptible (SS), and resistant by susceptible (RS) populations were available for bioassays at the same time. F1 larvae (RS) were also reared and crossed with resistant parents to obtain the backcross RS×RR. The procedures for this cross were identical to those described above. (Pereira et al. (2004) supra.)

Dose-mortality data were analyzed by probit regression to generate lethal concentrations and determine significance of differences among strains and generations. A likelihood ratio test was performed at α=0.05 to determine the significance of resistance ratios. Results from concentration-response bioassays using Bt endotoxins on unselected and selected Cry1F colonies showed that the selected colony exhibited high resistance to Cry1F compared to the unselected colony. Cross resistance studies showed that there is no cross resistance to Cry1Ab and Cry9C, although a low but significant (6.89-fold) cross resistance to Cry1Ac was detected. While not wishing to be bound by any one theory, this result suggests that Cry1Ac may share a common binding receptor with Cry1F. (Pereira et al. (2004) supra.)

Data obtained by Pereira et al. (2004) supra, clearly showed that Cry1F resistance is recessive as indicated by the progeny's dose-response curves, which are very similar to the unselected colony. The degree of dominance based on the LC₅₀ results was −0.78, which is close to −1 (completely recessive). In addition, there is no evidence of sex-linked heritance.

Furthermore, Pereira et al. (2004) supra showed that the response pattern of the backcross progeny indicated a single gene was responsible for the resistance. If resistance had been conferred by a single gene, the back cross generation would have consisted of a 1:1 ratio of RS:SS genotypes and a plateau at 50% mortality.

These results clearly show that the selected colony studied by Pereira et al. (2004) supra, exhibits high levels of resistance to Cry1F (>3000 fold) and is suitable for characterizing Cry1F resistance. In addition, these results indicate that the resistance mechanism is specific for Cry1F as there is only low cross resistance to Cry1Ac, which indicates that Cry1F and Cry1Ac share a common binding site. Cry1F resistance identified in this selected colony is monogenic, recessive and autosomal. This data supports a recessive pattern of inheritance for ECB resistance to Cry1F. Further, these results support the high-dose/refuge approach currently in place to manage resistance in ECB to Cry1F corn, which is dependent on a recessive inheritance pattern. Since selection of ECB to Cry1F resistance does not lead to loss of susceptibility to Cry1Ab and Cry9C, rotation with these toxins or their use in a multiple toxin approach appears to be a reasonable strategy to manage Cry1F resistance in Bt corn. In addition, the availability of this resistant strain will allow estimation of Cry1F resistance allele frequency in the field, and provides a tool to identify the biochemical and physiological basis of resistance and a means to develop molecular probes to monitor the evolution of resistance in the field. (Pereira et al. (2004) supra.)

Having illustrated and described the principles of the present invention, it should be apparent to persons skilled in the art that the invention can be modified in arrangement and detail without departing from such principles. We claim all modifications that are within the spirit and scope of the appended claims.

All publications and published patent documents cited in this specification are incorporated herein by reference to the same extent as if each individual publication or patent application was specifically and individually indicated to be incorporated by reference.

1. A method of reducing the development of resistant pests in a field of transgenic pest resistant crops comprising the steps of:
a) blending seed of a first transgenic pest resistant crop with seed of a second transgenic pest resistant crop to provide a seed mixture wherein said first pest resistant crop and said second pest resistant crop are pesticidal to the same target pest but through a different mode of pesticidal action; and
b) planting said seed mixture in a field wherein said seed mixture consists of from about 100% to about 0% of said first transgenic pest resistant crop and of from about 100% to about 0% of said second transgenic pest resistant crop.

2. The method of claim 1, wherein said same target pest is ECB.

3. The method of claim 1, wherein said same target pest is WCRW.

4. The method of claim 1, wherein said different mode of pesticidal action comprises binding without competition to different binding sites in the gut membranes of said same target pest.

5. The method of claim 1, further comprising treating said first transgenic pest resistant crop seed and said second transgenic pest resistant crop seed with a pesticidal agent selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, and carbamates.

6. The method of claim 1 wherein said first transgenic pest resistant crop produces a Cry1F protein and said second transgenic pest resistant crop produces a Cry1A(b) protein.

7. The method of claim 1 wherein said first transgenic pest resistant crop produces a Cry 34/35 protein and said second transgenic pest resistant crop produces a Cry 3 protein.

8. A method of reducing the development of resistant pests in a field of transgenic pest resistant crops comprising the steps of:
a) blending seed of a first transgenic pest resistant crop which contains one or more transgenes having pesticidal activity against a first target pest wherein said one or more transgenes are pesticidal to said first target pest through different modes of pesticidal action, with seed of a second transgenic pest resistant crop which contains one or more transgenes having pesticidal activity against a second target pest wherein said one or more transgenes are pesticidal to said second target pest through different modes of pesticidal action, to provide a seed mixture wherein said first pest resistant crop and said second pest resistant crop are pesticidal to different target pests; and
b) planting said seed mixture in a field wherein said seed mixture consists of from about 100% to about 0% of said first transgenic pest resistant crop and of from about 100% to about 0% of said second transgenic pest resistant crop.

9. The method of claim 8, wherein said first target pest is ECB.

10. The method of claim 8, wherein said second target pest is WCRW.

11. The method of claim 8, wherein said different modes of pesticidal action comprises binding without competition to different binding sites in the gut membranes of said first target pest and said second target pest.

12. The method of claim 8, further comprising treating said first transgenic pest resistant crop seed and said second transgenic pest resistant crop seed with a pesticidal agent selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, and carbamates.

13. The method of claim 8 wherein said first transgenic pest resistant crop produces a Cry 1F protein and a Cry 1A(b) protein and said second transgenic pest resistant crop produces a Cry 34/35 protein and a Cry 3 protein.

14. The method of claim 1, wherein said first transgenic pest resistant crop and said second transgenic pest resistant crop further contains a herbicide resistance gene selected from the group consisting of GAT and EPSPS.

15. The method of claim 8, wherein said first transgenic pest resistant crop and said second transgenic pest resistant crop further contains a herbicide resistance gene selected from the group consisting of GAT and EPSPS.

16. A method for deploying a transgenic pest resistant refuge crop into a field of a transgenic pest resistant crop comprising the steps of:
a) blending seed of a transgenic pest resistant refuge crop with seed of a transgenic pest resistant crop to provide a seed mixture wherein said pest resistant refuge crop and said pest resistant crop are pesticidal to the same target pest but through a different mode of pesticidal action; and
b) planting said seed mixture in a field wherein said seed mixture consists of from about 100% to about 0% of said transgenic pest resistant refuge crop and of from about 100% to about 0% of said transgenic pest resistant crop.

17. The method of claim 16, wherein said same target pest is ECB.

18. The method of claim 16, wherein said same target pest is WCRW.

19. The method of claim 16, wherein said different mode of pesticidal action comprises binding without competition to different binding sites in the gut membranes of said same target pest.

20. The method of claim 16, further comprising treating said transgenic pest resistant refuge crop seed and said transgenic pest resistant crop seed with a pesticidal agent selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, and carbamates.

21. The method of claim 16, wherein said transgenic pest resistant refuge crop produces a Cry 1F protein and said transgenic pest resistant crop produces a Cry 1A(b) protein.

22. The method of claim 16, wherein said transgenic pest resistant refuge crop produces a Cry 34/35 protein and said transgenic pest resistant crop produces a Cry 3 protein.

23. A method for deploying a transgenic pest resistant refuge crop into a field of a transgenic pest resistant crop comprising the steps of:
a) blending seed of a transgenic pest resistant refuge crop which contains one or more transgenes having pesticidal activity against a first target pest wherein said one or more transgenes are pesticidal to said first target pest through different modes of pesticidal action, with seed of a transgenic pest resistant crop which contains one or more transgenes having pesticidal activity against a second target pest wherein said one or more transgenes are pesticidal to said second target pest through different modes of pesticidal action, to provide a seed mixture wherein said pest resistant refuge crop and said pest resistant crop are pesticidal to different target pests; and
b) planting said seed mixture in a field wherein said seed mixture consists of from about 100% to about 0% of said transgenic pest resistant refuge crop and of from about 100% to about 0% of said transgenic pest resistant crop.

24. The method of claim 23, wherein said first target pest is ECB.

25. The method of claim 23, wherein said second target pest is WCRW.

26. The method of claim 23, wherein said different modes of pesticidal action comprises binding without competition to different binding sites in the gut membranes of said first target pest and said second target pest.

27. The method of claim 23, further comprising treating said first transgenic pest resistant crop seed and said second transgenic pest resistant crop seed with a pesticidal agent selected from the group consisting of pyrethrins and synthetic pyrethrins, oxadizines, chloronicotinyls, nitroguanidines, triazoles, organophosphates, pyrrols, pyrazoles, phenol pyrazoles, diacylhydrazines, biological/fermentation products, and carbamates.

28. The method of claim 23, wherein said first transgenic pest resistant crop produces a Cry 1F protein and a Cry 1A(b) protein and said second transgenic pest resistant crop produces a Cry 34/35 protein and a Cry 3 protein.

29. The method of claim 16, wherein said transgenic pest resistant refuge crop and said transgenic pest resistant crop further contains a herbicide resistance gene selected from the group consisting of GAT and EPSPS.

30. The method of claim 23, wherein said transgenic pest resistant refuge crop and said transgenic pest resistant crop further contains a herbicide resistance gene selected from the group consisting of GAT and EPSPS.

31. The method of claim 1 wherein said first transgenic pest resistant crop produces a Cry1F protein and said second transgenic pest resistant crop produces a Cry9 protein.

32. The method of claim 1 wherein said first transgenic pest resistant crop produces a Cry1Ab protein and said second transgenic pest resistant crop produces a Cry9 protein.

33. The method of claim 1 wherein said first transgenic pest resistant crop produces a Cry1Ab protein and said second transgenic pest resistant crop produces a Cry2 protein.

34. The method of claim 1 wherein said first transgenic pest resistant crop produces a Cry1F protein and said second transgenic pest resistant crop produces a Cry2 protein.

35. The method of claim 1 wherein said first transgenic pest resistant crop produces a Cry1A(b) protein and said second transgenic pest resistant crop produces a Cry2 and a Vip3A protein.

36. The method of claim 1 wherein said first transgenic pest resistant crop produces a Cry1F protein and said second transgenic pest resistant crop produces a Cry2 and a Vip3A protein.

37. The method of claim 1 wherein said first transgenic pest resistant crop produces a Cry1Ab protein and said second transgenic pest resistant crop produces a Cry1F and a Vip3A protein.

38. The method of claim 8 wherein said first transgenic pest resistant crop produces a Cry 1F protein and a Cry 1A(b) protein and said second transgenic pest resistant crop produces a Cry 34/35 protein and a Cry 3 protein.

39. The method of claim 8 wherein said first transgenic pest resistant crop produces a Cry 1F protein and a Cry 9 protein and said second transgenic pest resistant crop produces a Cry 34/35 protein and a Cry 3 protein.

40. The method of claim 8 wherein said first transgenic pest resistant crop produces a Cry 1A(b) protein and a Cry 1F protein and said second transgenic pest resistant crop produces a Cry 34/35 protein and a Cry 3 protein.

41. The method of claim 8 wherein said first transgenic pest resistant crop produces a Cry 1A(b) protein and a Cry 2 protein and said second transgenic pest resistant crop produces a Cry 34/35 protein and a Cry 3 protein.

42. The method of claim 8 wherein said first transgenic pest resistant crop produces a Cry 1F protein and a Cry 2 protein and said second transgenic pest resistant crop produces a Cry 34/35 protein and a Cry 3 protein.

43. The method of claim 8 wherein said first transgenic pest resistant crop produces a Cry 1A(b) protein, a Cry 2 protein and a Vip3A protein and said second transgenic pest resistant crop produces a Cry 34/35 protein and a Cry 3 protein.

44. The method of claim 8 wherein said first transgenic pest resistant crop produces a Cry 1F protein, a Cry 2 protein and a Vip3A protein and said second transgenic pest resistant crop produces a Cry 34/35 protein and a Cry 3 protein.

45. The method of claim 16 wherein said first transgenic pest resistant crop produces a Cry1F protein and said second transgenic pest resistant crop produces a Cry9 protein.

46. The method of claim 16 wherein said first transgenic pest resistant crop produces a Cry1Ab protein and said second transgenic pest resistant crop produces a Cry9 protein.

47. The method of claim 16 wherein said first transgenic pest resistant crop produces a Cry1Ab protein and said second transgenic pest resistant crop produces a Cry2 protein.

48. The method of claim 16 wherein said first transgenic pest resistant crop produces a Cry1F protein and said second transgenic pest resistant crop produces a Cry2 protein.

49. The method of claim 16 wherein said first transgenic pest resistant crop produces a Cry1A(b) protein and said second transgenic pest resistant crop produces a Cry2 and a Vip3A protein.

50. The method of claim 16 wherein said first transgenic pest resistant crop produces a Cry1F protein and said second transgenic pest resistant crop produces a Cry2 and a Vip3A protein.

51. The method of claim 16 wherein said first transgenic pest resistant crop produces a Cry1Ab protein and said second transgenic pest resistant crop produces a Cry1F and a Vip3A protein.

52. The method of claim 23 wherein said first transgenic pest resistant crop produces a Cry 1F protein and a Cry 1A(b) protein and said second transgenic pest resistant crop produces a Cry 34/35 protein and a Cry 3 protein.

53. The method of claim 23 wherein said first transgenic pest resistant crop produces a Cry 1F protein and a Cry 9 protein and said second transgenic pest resistant crop produces a Cry 34/35 protein and a Cry 3 protein.

54. The method of claim 23 wherein said first transgenic pest resistant crop produces a Cry 1A(b) protein and a Cry 1F protein and said second transgenic pest resistant crop produces a Cry 34/35 protein and a Cry 3 protein.

55. The method of claim 23 wherein said first transgenic pest resistant crop produces a Cry 1A(b) protein and a Cry 2 protein and said second transgenic pest resistant crop produces a Cry 34/35 protein and a Cry 3 protein.

56. The method of claim 23 wherein said first transgenic pest resistant crop produces a Cry 1F protein and a Cry 2 protein and said second transgenic pest resistant crop produces a Cry 34/35 protein and a Cry 3 protein.

57. The method of claim 23 wherein said first transgenic pest resistant crop produces a Cry 1A(b) protein, a Cry 2 protein and a Vip3A protein and said second transgenic pest resistant crop produces a Cry 34/35 protein and a Cry 3 protein.

58. The method of claim 23 wherein said first transgenic pest resistant crop produces a Cry 1F protein, a Cry 2 protein and a Vip3A protein and said second transgenic pest resistant crop produces a Cry 34/35 protein and a Cry 3 protein.
